(19) United States
(12) Patent Application Publication
(10) Pub. No.: US 2011/0319502 A1
(43) Pub. Date: Dec. 29, 2011

(54) Compositions and Methods for Enhancing Reduction of Spore-Forming Microorganisms

(76) Inventors: Martin J. Coffey, Pittsford, NY (US); Mohannad Shawer, Webster, NY (US)

(21) Appl. No.: 13/116,100

(22) Filed: May 26, 2011

Related U.S. Application Data
(60) Provisional application No. 61/358,463, filed on Jun. 25, 2010.

(51) Int. Cl. A61K 47/26; A61K 47/18

(52) U.S. Cl. 514/777; 514/784

(57) ABSTRACT

A pharmaceutical formulation comprises a pharmaceutically acceptable preservative and a material selected from the group consisting of D-glucose, sucrose, maltose, D-mannose, trehalose, glutamic acid, mixtures thereof, and combinations thereof, wherein the formulation has enhanced preservative efficacy against spore-forming microorganisms. The formulation can further comprise boric acid and/or phosphate. A method for enhancing the preservative efficacy of a pharmaceutical formulation against spore-forming microorganisms, comprising adding a pharmaceutically acceptable preservative, and a material selected from the group consisting of D-glucose, sucrose, maltose, D-mannose, trehalose, glutamic acid, mixtures thereof, and combinations thereof, to the pharmaceutical formulation. The formulation optionally includes an active agent for treating or controlling a disease or disorder of the eye. The formulation may be used to treat, clean, disinfect, store, wet, or rewet contact lenses.

### CROSS REFERENCE

This application claims the benefit of Provisional Patent Application No. 61/358,463 filed Jun. 25, 2010, which is incorporated by reference herein.

### BACKGROUND

The present invention relates to compositions and methods for enhancing reduction of spore-forming microorganisms. In particular, the present invention relates to a method for enhancing the preservative efficacy of pharmaceutical compositions against spore-forming microorganisms.

Pharmaceutical formulations are commonly provided in multi-use bottles. Formulations, such as ophthalmic compositions, find uses in many ophthalmic applications. These compositions are often instilled directly into the eye one or more times a day to either deliver medications or to relieve symptoms of eye conditions, such as dry eye or inflammation of the superficial tissues of the eye accompanying various allergic reactions (such as hay fever allergies and the like, irritation of the eye due to foreign bodies, or eye fatigue). Other ophthalmic solutions are employed in the field of contact-lens care. Contact-lens solutions are utilized to soak, disinfect, clean, and wet contact lenses. These solutions are not instilled directly in the eye from the bottle, but do subsequently come into contact with the eye when the lenses are placed on the eye.

Ophthalmic compositions are provided sterile, but once opened, are susceptible to microbial contamination. In the case of multi-use solutions, the formulations contain at least a preservative designed to kill microorganisms that come in contact with the solution, protecting the patient from infection due to a contaminated ophthalmic solution during the prescribed usage.

Typically, preservatives for ophthalmic compositions fall into two traditional categories: alcohols and amines or ammonium-containing compounds. Typical alcohol-based anti-microbial agents include benzyl alcohol, phenethyl alcohol, and chlorbutanol. Alcohol-based preservatives work by disorganizing the lipid structure of cell membrane, and thus increase permeability of the cell wall, leading to cell lysis. These alcohols have limited solubility in aqueous solutions and tend not to be stable preservatives due to being susceptible to oxidation, evaporation, and interaction with the plastic bottle. More commonly, organic amines and ammonium-containing compounds are utilized as anti-microbial agents in ophthalmic solutions. Representative compounds in this category include benzalkonium chloride (“BAK”), benzododecinium bromide (“BDD”), chlorhexidine, polymeric biguanide (such as polyhexamethylene biguanide or “PHMB”). It is believed that the electrophilicity of the nitrogen-containing moieties of these compounds promotes their interaction with the negatively charged cell membranes of the microorganisms, leading to cell lysis, and thus severely impacting their survival.

Although amines and ammonium-containing compounds have good anti-microbial activity, and are used commercially to preserve ophthalmic solutions, there are significant disadvantages associated with these compounds. In particular, these compounds used at higher doses can be toxic to the sensitive tissues of the eye. For example, BAK-containing ophthalmic solutions are known to cause eye irritation in patients. It causes growth arrest at very low concentration (0.00001%), apoptosis at 0.01%, and necrosis at higher concentrations (0.05-0.1%). Patients who may be at greater risk of BAK-induced adverse effects are those with dry-eye syndrome since they often need to use eye drop over an extended period of time. Polymeric amines and ammonium-containing compounds are less toxic than BAK but still can cause irritation responses in some other patients. For example, polyquaternium-1 (α-4-{tris(2hydroxyethyl)ammonium-2-butenyl}poly{1-dimethylammonium-2-butenyl}-ω-tris(2-hydroxyethyl)ammonium chloride), also known as Polyquad®, has been shown to be less toxic than BAK and used in a limited number of ophthalmic formulations. However, polyquaternium-1 still shows some adverse effects on ocular tissues. A 0.5% polyquaternium-1 formulation has been shown significantly to decrease goblet cell density. Healthy goblet cells are required to produce adequate mucin, which is one of three component layers of the tear film. A. Labbé et al., J. Ocular Pharmacol. & Therapeutics, Vol. 22, No. 4, 267 (2006). Chlorhexidine, on the other hand, has proven to be more biocompatible than the other amines and ammonium-containing anti-microbial agents and, therefore, non-irritating at the levels typically used. However, the mildness of chlorhexidine to the ocular environment is offset by the fact that chlorhexidine is a relatively weak preservative.

Oxidative preservatives, which work by oxidizing cell walls or membranes, affecting membrane-bound enzymes, and disrupting cellular function. U.S. Pat. Nos. 5,576,028; 5,607,698; 5,725,887; and 5,807,585 and European Patent 035486 disclose solutions, which may be ophthalmic solutions or contact lens solutions, containing from 10 ppm (0.001%) to 1000 ppm (0.1%) hydrogen peroxide and a hydrogen peroxide stabilizer. However, the long-term preservative efficacy of these solutions is not known. It is suggested in these patents that hydrogen peroxide concentration should be in trace amounts in order to be tolerable to the patient upon direct application.

The amounts of these traditional preservatives used in pharmaceutical compositions to ensure their sterility in multi-dose containers often render these compositions uncomfortable when instilled in the eyes of sensitive patients.

Therefore, there is a continued need to provide improved pharmaceutical compositions having enhanced preservative efficacy against a range of microorganisms and methods for enhancing the preservative efficacy of pharmaceutical compositions.

### SUMMARY

In general, the present invention provides improved pharmaceutical compositions or formulations having enhanced preservative efficacy against spore-forming microorganisms, and methods for enhancing the preservative efficacy of pharmaceutical compositions against spore-forming microorganisms.

In one aspect, such spore-forming microorganisms are selected from the group consisting of spore-forming bacteria, spore-forming fungi, and combinations thereof.

In another aspect, the compositions and methods of the present invention are also effective against non-spore-forming microorganisms.

In still another such compositions or formulations provide improved safety and/or comfort to the users.

In still another aspect, such compositions and methods are effective in adversely affecting the viability of spore-forming microorganisms or in inhibiting their growth and provide better safety and/or comfort to the users.

In still another aspect, a pharmaceutical formulation of the present invention comprises a pharmaceutically acceptable preservative and a preservative efficacy-enhancing material selected from the group consisting of D-glucose, sucrose, maltose, D-mannose, trehalose, glutamic acid, mixtures thereof, and combinations thereof.

In yet another aspect, a pharmaceutical formulation of the present invention comprises a pharmaceutically acceptable preservative, boric acid, and a preservative efficacy-enhancing material selected from the group consisting of D-glucose, sucrose, maltose, D-mannose, trehalose, glutamic acid, mixtures thereof, and combinations thereof.

In still another aspect, a pharmaceutical formulation of the present invention comprises a pharmaceutically acceptable preservative, phosphate, boric acid, and a material selected from the group consisting of D-glucose, sucrose, maltose, D-mannose, trehalose, glutamic acid, mixtures thereof, and combinations thereof.

In a further aspect, such a pharmaceutical formulation is an ophthalmic composition, which results in less irritation when applied to ocular tissues surfaces than prior-art compositions.

In still another aspect, representatives of such spore-forming fungi comprise Aspergillus species, such as Aspergillus brasiliensis.

In a further aspect, the present invention provides a method for making a pharmaceutical formulation having enhanced preservative efficacy against spore-forming microorganisms. The method comprises adding a pharmaceutically acceptable preservative and a material selected from the group consisting of D-glucose, sucrose, maltose, D-mannose, trehalose, glutamic acid, mixtures thereof, and combinations thereof, to a pharmaceutical formulation.

In yet another aspect, the present invention provides a method for treating, controlling, or preventing a condition or disorder of an eye. The method comprises topically administering to the eye an effective amount of an ophthalmic composition that comprises a pharmaceutically acceptable preservative and a material selected from the group consisting of D-glucose, sucrose, maltose, D-mannose, trehalose, glutamic acid, mixtures thereof, and combinations thereof.

In a further aspect, the present invention provides a method for treating an ophthalmic device. The method comprises contacting the ophthalmic device with an ophthalmic solution comprising a pharmaceutically acceptable preservative and a material selected from the group consisting of D-glucose, sucrose, maltose, D-mannose, trehalose, glutamic acid, mixtures thereof, and combinations thereof.

In still a further aspect, the ophthalmic device is a contact lens.

Other features and advantages of the present invention will become apparent from the following detailed description and claims.

### DETAILED DESCRIPTION

In general, the present invention provides improved pharmaceutical compositions or formulations having enhanced preservative efficacy against spore-forming microorganisms, and methods for enhancing the preservative efficacy of pharmaceutical compositions against spore-forming microorganisms.

In one aspect, the improved pharmaceutical compositions or formulations and methods provide improved safety and/or comfort to the users.

In another aspect, the present invention provides an ophthalmic composition that is effective in adversely affecting the viability of spore-forming microorganisms or in inhibiting their growth therein, methods of making, and methods of using such composition. In addition, the composition is also effective in adversely affecting the viability of non-spore-forming microorganisms or in inhibiting their growth therein. Within the scope of the present invention, the microorganisms that are adversely affected by a formulation of the present invention include microorganisms selected from the group consisting of bacteria, yeasts, molds, and mixtures thereof.

In one aspect, pharmaceutical compositions or formulations of the present invention can kill or adversely affect the survival or propagation of such microorganisms when they are in contact with such compositions or formulations, such as to result in reduction of their population therein. In one embodiment, representatives of such microorganisms comprise Staphylococcus aureus (or S. aureus), Pseudomonas aeruginosa (or P. aeruginosa), Eschrechia coli (or E. coli), Candida albicans (or C. albicans), and Aspergillus brasiliensis (or A. brasiliensis).

In another aspect, a pharmaceutical composition or formulation of the present invention comprises a pharmaceutically acceptable preservative and a preservative efficacy-enhancing material selected from the group consisting of D-glucose, sucrose, maltose, D-mannose, trehalose, glutamic acid, mixtures thereof, and combinations thereof.

A suitable pharmaceutically acceptable preservative is selected from the group consisting of pharmaceutically acceptable alcohols, amines and ammonium-containing compounds, hydrogen peroxide and compounds that produce hydrogen peroxide in said composition (such as carbamide peroxide, carbamide perhydrate, percarbamide, or perborate salts), oxychloro compounds such as chlorine dioxide, zinc compounds, mixtures thereof, and combinations thereof.

In another aspect, the pharmaceutically acceptable preservative is include in a composition of the present invention in an amount that does not produce irritation or discomfort to an eye of an average patient when such composition is administered thereto.

Non-limiting examples of ammonium-containing compounds include benzalkonium chloride (“BAK”), benzododecinium bromide (“BDD”). chlorhexidine, polymeric biguanide (such as polyhexamethylene biguanide or “PHMB”), polyquaternium-1 (also known as polidronium chloride, formula shown below), polyquaternium-4 (hydroxyethylcellulose dimethyl-diallyl ammonium chloride copolymer, sometimes known under the tradename of Celquat® H-100 or Celquat® L-200), and polyquaternium-42 (formula shown below).

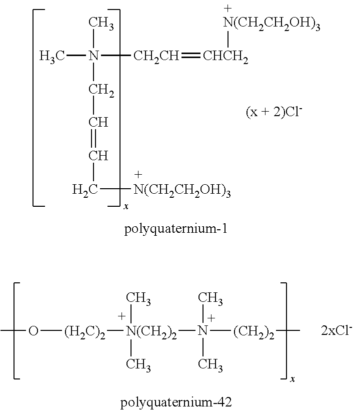

Other polyquaternium compounds, which are described in International Cosmetic Ingredient Dictionary and Handbook, can also be used in a composition of the present invention. Non-limiting examples of such other polyquaternium compounds are polyquaternium-2, -5, -6, -7, -8, -9, -45, -54, -71, and -72.

Typical alcohol-based anti-microbial agents include benzyl alcohol, phenethyl alcohol, and chlorbutanol.

In one embodiment, a composition of the present invention is free of a material selected from the group consisting of organic nitrogen-containing compounds containing a plurality of positive charges, such as organic nitrogen-containing small molecules or polymers or alcohols containing a plurality of positive charges.

In one aspect, a pharmaceutical composition of the present invention comprises a pharmaceutical active ingredient, a pharmaceutically acceptable preservative and a preservative efficacy-enhancing material selected from the group consisting of D-glucose, sucrose, maltose, D-mannose, trehalose, glutamic acid, mixtures thereof, and combinations thereof, wherein the pharmaceutical composition has an enhanced preservative efficacy against a spore-forming microorganism.

In one embodiment, any one of the pharmaceutical compositions of the present invention herein disclosed further comprises boric acid.

In another embodiment, a pharmaceutical composition of the present invention comprises a pharmaceutical active ingredient, a pharmaceutically acceptable preservative, boric acid, phosphate, and a preservative efficacy-enhancing material selected from the group consisting of D-glucose, sucrose, maltose, D-mannose, trehalose, glutamic acid, mixtures thereof, and combinations thereof, wherein the pharmaceutical composition has an enhanced preservative efficacy against a spore-forming microorganism.

A pharmaceutical composition or formulation of the present invention comprises a pharmaceutical active ingredient, a pharmaceutically acceptable preservative, boric acid, phosphate, and a preservative efficacy-enhancing material selected from the group consisting of D-glucose, sucrose, mixtures thereof, and combinations thereof, wherein the pharmaceutical composition has an enhanced preservative efficacy against a spore-forming microorganism.

A pharmaceutical composition or formulation of the present invention comprises a pharmaceutical active ingredient, a pharmaceutically acceptable preservative, boric acid, phosphate, and a preservative efficacy-enhancing material selected from the group consisting of D-glucose, sucrose, mixtures thereof, and combinations thereof, wherein the preservative efficacy-enhancing material enhances preservative efficacy against a spore-forming microorganism.

In one aspect, the spore-forming microorganism against which any one of the compositions of the present invention herein disclosed is effective is a spore-forming mold or yeast.

In another aspect, the spore-forming microorganism against which any one of the compositions or formulations of the present invention herein disclosed is effective is a spore-forming A. brasiliensis.

Any one of the pharmaceutical compositions or formulations of the present invention herein disclosed can comprise a pharmaceutical active ingredient, a pharmaceutically acceptable preservative, boric acid, phosphate, and a preservative efficacy-enhancing material selected from the group consisting of D-glucose, wherein the pharmaceutical composition has an enhanced preservative efficacy against a spore-forming microorganism.

Any one of the pharmaceutical compositions or formulations of the present invention herein disclosed can comprise a pharmaceutical active ingredient, a pharmaceutically acceptable preservative, boric acid, phosphate, and a preservative efficacy-enhancing material selected from the group consisting of D-glucose, sucrose, and mixtures thereof; wherein the pharmaceutical composition has an enhanced preservative efficacy against a spore-forming microorganism, the pharmaceutically acceptable preservative is selected from the group consisting of pharmaceutically acceptable alcohols, amines and ammonium-containing compounds, hydrogen peroxide and compounds that produce hydrogen peroxide in said composition (such as carbamide peroxide, carbamide perhydrate, percarbamide, or perborate salts), oxychloro compounds such as chlorine dioxide, zinc compounds, mixtures thereof, and combinations thereof.

Any one of the pharmaceutical compositions or formulations of the present invention can comprise a pharmaceutical active ingredient, a pharmaceutically acceptable preservative, boric acid, phosphate, and a preservative efficacy-enhancing material selected from the group consisting of D-glucose, sucrose, maltose, D-mannose, trehalose, glutamic acid, mixtures thereof, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of pharmaceutically acceptable alcohols, amines and ammonium-containing compounds, hydrogen peroxide and compounds that produce hydrogen peroxide in said composition (such as carbamide peroxide, carbamide perhydrate, percarbamide, or perborate salts), oxychloro compounds such as chlorine dioxide, zinc compounds, mixtures thereof, and combinations thereof.

Any one of the pharmaceutical compositions or formulations of the present invention can comprise a pharmaceutical active ingredient, a pharmaceutically acceptable preservative, boric acid, phosphate, and a preservative efficacy-enhancing material selected from the group consisting of D-glucose, sucrose, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of polyquaternium-1, -2, -4, -5, -6, -7, -8, -9, -45, -54, -71, and -72.

Any one of the pharmaceutical compositions or formulations of the present invention can comprise a pharmaceutical active ingredient, a pharmaceutically acceptable preservative, boric acid, phosphate, and a preservative efficacy-enhancing material selected from the group consisting of D-glucose, sucrose, maltose, D-mannose, trehalose, glutamic acid, mixtures thereof, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of polyquaternium-1, -2, -4, -5, -6, -7, -8, -9, -45, -54, -71, and −72.

Any one of the pharmaceutical compositions or formulations of the present invention can comprise a pharmaceutical active ingredient, a pharmaceutically acceptable preservative, a buffer, and a preservative efficacy-enhancing material selected from the group consisting of D-glucose, sucrose, maltose, D-mannose, trehalose, glutamic acid, mixtures thereof, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of polyquaternium-1, -2, -4, -5, -6, -7, -8, -9, -45, -54, -71, and -72.

Any one of the pharmaceutical compositions or formulations of the present invention can comprise a pharmaceutical active ingredient, a pharmaceutically acceptable preservative, boric acid, phosphate, and a preservative efficacy-enhancing material selected from the group consisting of D-glucose, sucrose, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of polyquaternium-1, -2, -4, -5, -6, -7, -8, -9, -45, -54, -71, and -72.

Any one of the pharmaceutical compositions or formulations of the present invention can comprise a pharmaceutical active ingredient, a pharmaceutically acceptable preservative, and a preservative efficacy-enhancing material selected from the group consisting of D-glucose, sucrose, maltose, D-mannose, trehalose, glutamic acid, mixtures thereof, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of polyquaternium-1, -2, -4, -5, -6, -7, -8, -9, -45, -54, -71, and -72.

Any one of the pharmaceutical compositions or formulations of the present invention can comprise a pharmaceutical active ingredient, a pharmaceutically acceptable preservative, and a preservative efficacy-enhancing material selected from the group consisting of D-glucose, sucrose, maltose, D-mannose, trehalose, glutamic acid, mixtures thereof, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of a source of hydrogen peroxide (such as perborate, peracetate, or urea peroxide), hydrogen peroxide, stabilized oxychloro complex, and mixtures thereof.

Any one of the pharmaceutical compositions or formulations of the present invention can comprise a pharmaceutical active ingredient, a buffer (such as borate, phosphate, or mixtures thereof), a pharmaceutically acceptable preservative, and a preservative efficacy-enhancing material selected from the group consisting of D-glucose, sucrose, maltose, D-mannose, trehalose, glutamic acid, mixtures thereof, and combinations thereof, wherein the pharmaceutical composition or formulation has an enhanced preservative efficacy against a spore-forming microorganism; wherein the pharmaceutical composition or formulation has an enhanced preservative efficacy against a spore-forming microorganism, the pharmaceutically acceptable preservative is selected from the group consisting of a source of hydrogen peroxide (such as perborate, peracetate, or urea peroxide), hydrogen peroxide, stabilized oxychloro complex, and mixtures thereof.

Any one of the pharmaceutical compositions or formulations of the present invention can comprise a pharmaceutical active ingredient, a buffer (such as borate, phosphate, or mixtures thereof), a pharmaceutically acceptable preservative, and a preservative efficacy-enhancing material selected from the group consisting of D-glucose, sucrose, mixtures thereof, and combinations thereof, wherein the pharmaceutical composition or formulation has an enhanced preservative efficacy against a spore-forming microorganism: wherein the pharmaceutical composition or formulation has an enhanced preservative efficacy against a spore-forming microorganism, the pharmaceutically acceptable preservative is selected from the group consisting of a source of hydrogen peroxide (such as perborate, peracetate, or urea peroxide), hydrogen peroxide, stabilized oxychloro complex, and mixtures thereof.

Any one of the pharmaceutical compositions or formulations of the present invention can be in the form of a solution, a suspension, an emulsion, a dispersion, an ointment, or a cream.

Any one of the pharmaceutical compositions or formulations of the present invention is in the form of, or can comprise, a solution or a suspension.

Any one of the pharmaceutical compositions or formulations can be in the form of, or can comprise, an aqueous solution.

Procedure for evaluating the preservative efficacy (“PE”) of a pharmaceutical formulation of the present invention against microorganisms

The microorganisms against which the PE of a pharmaceutical formulation of the present invention is evaluated are S. aureus, E. coli, P. aeruginosa, C. albicans, and A. brasiliensis. This procedure applies to the US FDA premarket notification (510(k)) guidance document and USP/ISO/DIS 14730 standard preservative efficacy testing with a 14-day rechallenge. The evaluations were conducted with 3 separate lots of each test solution for each microorganism. Each lot was tested with a different preparation of each microorganism.

Bacterial cells were grown on Tryptic Soy Agar (“TSA”) slants at a temperature in the range from 30 to 35° C. in an incubator for a time period from 18 to 24 hours. Fungal cells were grown on Sabouraud Dextrose Agar (“SDA”) slants at a temperature in the range from 20° C. to 25° C. in an incubator for a time period of 2 to 7 days. Cells were harvested in saline solution (5-10 ml, USP, 0.9% saline, with or without 0.1% Tween 80 surfactant, which was added to each agar slant, followed by gentle agitation with a sterile cotton swab. The cell suspensions were aseptically dispensed into separate sterile polypropylene centrifuge tubes. Cells were harvested by centrifugation at 3000 rpm for 10 minutes, washed one time, and suspended in Saline TS to a concentration of 2×10⁸ cells per ml.

The cell suspension (0.1 ml) was diluted with 20 ml of the test solution to reach a final concentration of from 1.0×10⁵ to 1.0×10⁶ colony-forming units (“CFU”). Phosphate Buffered Saline (“PBS”) was used as a control solution. The inoculated test and control solutions were incubated at a temperature ranging from 20° C. to 25° C. in static culture. At time zero, 1 ml of PBS (USP, pH 7.2) from the control solution was diluted with 9 ml of PBS and serially diluted cells were plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20 to 25° C. for a period ranging from 2 to 7 days.

Similarly, at days 7 and 14, a one-milliliter volume from a test solution was added into 9 ml of Dey-Engley neutralizing broth (“DEB”) and serially diluted in DEB and plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20° C. to 25° C. for a period ranging from 2 to 7 days. Developing colonies were counted.

Immediately following the day 14 sampling, test solutions were re-inoculated to give final concentrations of from 1.0×10⁴ to 1.0×10⁵ of each microorganism. At time zero, 1 ml from the inoculum control was added to 9 ml of PBS and subsequent serial dilutions were plated in triplicate on TSA for bacteria and SDA for fungi. The bacterial plates were incubated at a temperature ranging from 30 to 35° C. for a period ranging from 2 to 4 days. Fungal plates were incubated at a temperature ranging from 20 to 25° C. for a period ranging from 2 to 7 days.

At days 21 and 28, 1 ml from the test articles was added to 9 ml of DEB and again, serial dilutions were plated in triplicate on TSA. Plates were incubated at a temperature ranging from 30 to 35° C. for a period ranging from 2 days to 4 days and developing colonies counted.

Based on the acceptance criteria for bacteria for US Pharmacopeia (“USP”), a solution is acceptable if the concentration of viable bacteria, recovered per milliliter, is reduced by at least 1 log (log to the base 10 or log₁₀) at day 7, by at least 3 logs at day 14, and after a rechallenge at day 14, the concentration of bacteria is reduced by at least 3 logs by day 28. In addition, the solution is acceptable if the concentration of viable yeasts and molds, recovered per milliliter of the solution, remains at or below the initial concentration (within an experimental uncertainty of ±0.5 log) at day 14, and after a rechallenge at day 14, the concentration of viable yeasts and molds remains at or below the initial concentration (within an experimental uncertainty of ±0.5 log) at day 28.

It is notable that the acceptance criteria for a product marketed in Europe are more stringent than those stated above. A pharmaceutical composition meeting such more stringent criteria may be termed “having enhanced preservative efficacy against micro organisms.”

Based on a set of more stringent target acceptance criteria (“EP-A” or European target criteria) for bacteria, a solution is acceptable if the concentration of viable bacteria, recovered per milliliter, is reduced by at least 2 logs (log₁₀) at the end of 6 hours, at least 3 logs at the end of 24 hours, and after a rechallenge at day 14, no bacteria are recovered concentration (“no recovery,” considered to be equal to or greater than 4 logs reduction) by day 28. In addition, the solution is acceptable if the concentration of viable yeasts and molds, recovered per milliliter of the solution, is reduced by at least 2 logs by day 7, and after a rechallenge at day 14, the concentration of viable yeasts and molds remains at or below the initial concentration (within an experimental uncertainty of ±0.5 log) at day 28.

Based on an alternative set of more stringent acceptance criteria (“EP-B” or European acceptable criteria) for bacteria, a solution is acceptable if the concentration of viable bacteria, recovered per milliliter, is reduced by at least 1 log (log₁₀) at the end of 24 hours, at least 3 logs by day 7, and after a rechallenge at day 14, the concentration of bacteria remains at or below the initial concentration (within an experimental uncertainty of ±0.5 log) by day 28. In addition, the solution is acceptable if the concentration of viable yeasts and molds, recovered per milliliter of the solution, is reduced by at least 1 log by day 14, and after a rechallenge at day 14, the concentration of viable yeasts and molds remains at or below the initial concentration (within an experimental uncertainty of ±0.5 log) at day 28.

The foregoing acceptance criteria are summarized in Table 1.

TABLE 1
Preservative Efficacy Acceptance Criteria
Log₁₀ Reduction

| Time | 6 hour | 24 hour | 7 day | 14 day | 28 day |
|---|---|---|---|---|---|
| USP: | — | — | 1 | 3 | No |
| bacteria | | | | | increase |
| EP-A: | 2 | 3 | — | — | No |
| bacteria | | | | | recovery |
| EP-B: | — | 1 | 3 | — | No |
| bacteria | | | | | increase |
| USP: | — | — | No | No | No |
| fungi | | | increase | increase | increase |
| EP-A: | — | — | 2 | — | No |
| fungi | | | | | increase |
| EP-B: | — | — | — | 1 | No |
| fungi | | | | | increase |

“—” means “not required”

Furthermore, an ophthalmic solution of the present invention can comprise an active pharmaceutical ingredient (or therapeutic agent) such as anti-inflammatory agents, antibiotics, immunosuppressive agents, antiviral agents, antifungal agents, antiprotozoal agents, combinations thereof, or mixtures thereof. Non-limiting examples of anti-inflammatory agents include glucocorticosteroids (e.g., for short-term treatment) and non-steroidal anti-inflammatory drugs (“NSAIDs”).

Non-limiting examples of the glucocorticosteroids are: 21-acetoxypregnenolone, alclometasone, algestone, amcinonide, beclomethasone, betamethasone, budesonide, chloroprednisone, clobetasol, clobetasone, clocortolone, cloprednol, corticosterone, cortisone, cortivazol, deflazacort, desonide, desoximetasone, dexamethasone, diflorasone, diflucortolone, difluprednate, enoxolone, fluazacort, flucloronide, flumethasone, flunisolide, fluocinolone acetonide, fluocinonide, fluocortin butyl, fluocortolone, fluorometholone, fluperolone acetate, fluprednidene acetate, fluprednisolone, flurandrenolide, fluticasone propionate, formocortal, halcinonide, halobetasol propionate, halometasone, halopredone acetate, hydrocortarnate, hydrocortisone, loteprednol etabonate, mazipredone, medrysone, meprednisone, methylprednisolone, mometasone furoate, paramethasone, prednicarbate, prednisolone, prednisolone 25-diethylamino-acetate, prednisolone sodium phosphate, prednisone, prednival, prednylidene, rimexolone, tixocortol, triamcinolone, triamcinolone acetonide, triamcinolone benetonide, triamcinolone hexacetonide, their physiologically acceptable salts, derivatives thereof, combinations thereof, and mixtures thereof. In one embodiment, the therapeutic agent is selected from the group consisting of difluprednate, loteprednol etabonate, prednisolone, combinations thereof, and mixtures thereof.

Non-limiting examples of the NSAIDs are: aminoarylcarboxylic acid derivatives (e.g., enfenamic acid, etofenamate, flufenamic acid, isonixin, meclofenamic acid, mefenamic acid, niflumic acid, talniflumate, terofenamate, tolfenamic acid), arylacetic acid derivatives (e.g., aceclofenac, acemetacin, alclofenac, amfenac, amtolmetin guacil, bromfenac, bufexamac, cinmetacin, clopirac, diclofenac sodium, etodolac, felbinac, fenclozic acid, fentiazac, glucametacin, ibufenac, indomethacin, isofezolac, isoxepac, lonazolac, metiazinic acid, mofezolac, oxametacine, pirazolac, proglumetacin, sulindac, tiaramide, tolmetin, tropesin, zomepirac), arylbutyric acid derivatives (e.g., bumadizon, butibufen, fenbufen, xenbucin), arylcarboxylic acids (e.g., clidanac, ketorolac, tinoridine), arylpropionic acid derivatives (e.g., alminoprofen, benoxaprofen, bermoprofen, bucloxic acid, carprofen, fenoprofen, flunoxaprofen, flurbiprofen, ibuprofen, ibuproxam, indoprofen, ketoprofen, loxoprofen, naproxen, oxaprozin, piketoprolen, pirprofen, pranoprofen, protizinic acid, suprofen, tiaprofenic acid, ximoprofen, zaltoprofen), pyrazoles (e.g., difenamizole, epirizole), pyrazolones (e.g., apazone, benzpiperylon, feprazone, mofebutazone, morazone, oxyphenbutazone, phenylbutazone, pipebuzone, propyphenazone, ramifenazone, suxibuzone, thiazolinobutazone), salicylic acid derivatives (e.g., acetaminosalol, aspirin, benorylate, bromosaligenin, calcium acetylsalicylate, diflunisal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide o-acetic acid, salicyl sulfuric acid, salsalate, sulfasalazine), thiazinecarboxamides (e.g., ampiroxicam, droxicam, isoxicam, lornoxicam, piroxicam, tenoxicam), 8-acetamidocaproic acid, S-(5′-adenosyl)-L-methionine, 3-amino-4-hydroxybutyric acid, amixetrine, bendazac, benzydamine, bisabolol, bucolome, difenpiramide, ditazol, emorfazone, fepradinol, guaiazulene, nabumetone, nimesulide, oxaceprol, paranyline, perisoxal, proquazone, superoxide dismutase, tenidap, zileuton, their physiologically acceptable salts, combinations thereof, and mixtures thereof.

Non-limiting examples of antibiotics include doxorubicin; aminoglycosides (e.g., amikacin, apramycin, arbekacin, bambermycins, butirosin, dibekacin, dihydrostreptomycin, fortimicin(s), gentamicin, isepamicin, kanamycin, micronomicin, neomycin, neomycin undecylenate, netilmicin, paromomycin, ribostamycin, sisomicin, spectinomycin, streptomycin, tobramycin, trospectomycin), amphenicols (e.g., azidamfenicol, chloramphenicol, florfenicol, thiamphenicol), ansamycins (e.g., rifamide, rifampin, rifamycin SV, rifapentine, rifaximin), β-lactams (e.g., carbacephems (e.g., loracarbef)), carbapenems (e.g., biapenem, imipenem, meropenem, panipenem), cephalosporins (e.g., cefaclor, cefadroxil, cefamandole, cefatrizine, cefazedone, cefazolin, cefcapene pivoxil, cefclidin, cefdinir, cefditoren, cefepime, cefetamet, cefixime, cefinenoxime, cefodizime, cefonicid, cefoperazone, ceforamide, cefotaxime, cefotiam, cefozopran, cefpimizole, cefpiramide, cefpirome, cefpodoxime proxetil, cefprozil, cefroxadine, cefsulodin, ceftazidime, cefteram, ceftezole, ceftibuten, ceftizoxime, ceftriaxone, cefuroxime, cefuzonam, cephacetrile sodium, cephalexin, cephaloglycin, cephaloridine, cephalosporin, cephalothin, cephapirin sodium, cephradine, pivcefalexin), cephamycins (e.g., cefbuperazone, cefinetazole, cefininox, cefotetan, cefoxitin), monobactams (e.g., aztreonam, carumonam, tigemonam), oxacephems, flomoxef, moxalactam), penicillins (e.g., amdinocillin, amdinocillin pivoxil, amoxicillin, ampicillin, apalcillin, aspoxicillin, azidocillin, azlocillin, bacampicillin, benzylpenicillinic acid, benzylpenicillin sodium, carbenicillin, carindacillin, clometocillin, cloxacillin, cyclacillin, dicloxacillin, epicillin, fenbenicillin, floxacillin, hetacillin, lenampicillin, metampicillin, methicillin sodium, mezlocillin, nafcillin sodium, oxacillin, penamecillin, penethamate hydriodide, penicillin G benethamine, penicillin G benzathine, penicillin G benzhydrylamine, penicillin G calcium, penicillin G hydrabamine, penicillin G potassium, penicillin G procaine, penicillin N, penicillin O, penicillin V, penicillin V benzathine, penicillin V hydrabamine, penimepicycline, phenethicillin potassium, piperacillin, pivampicillin, propicillin, quinacillin, sulbenicillin, sultamicillin, talampicillin, temocillin, ticarcillin), lincosamides (e.g., clindamycin, lincomycin), macrolides (e.g., azithromycin, carbomycin, clarithromycin, dirithromycin, erythromycin, erythromycin acistrate, erythromycin estolate, erythromycin glucoheptonate, erythromycin lactobionate, erythromycin propionate, erythromycin stearate, josamycin, leucomycins, midecamycins, miokamycin, oleandomycin, primycin, rokitamycin, rosaramicin, roxithromycin, spiramycin, troleandomycin), polypeptides (e.g., amphomycin, bacitracin, capreomycin, colistin, enduracidin, enviomycin, fusafungine, gramicidin S, gramicidin(s), mikamycin, polymyxin, pristinamycin, ristocetin, teicoplanin, thiostrepton, tuberactinomycin, tyrocidine, tyrothricin, vancomycin, viomycin, virginiamycin, zinc bacitracin), tetracyclines (e.g., apicycline, chlortetracycline, clomocycline, demeclocycline, doxycycline, guamecycline, lymecycline, meclocycline, methacycline, minocycline, oxytetracycline, penimepicycline, pipacycline, rolitetracycline, sancycline, tetracycline), and others (e.g., cycloserine, mupirocin, tuberin).

Other examples of antibiotics are the synthetic antibacterials, such as 2,4-diaminopyrimidines (e.g., brodimoprim, tetroxoprim, trimethoprim), nitrofurans (e.g., furaltadone, furazolium chloride, nifuradene, nifuratel, nifurfoline, nifurpirinol, nifurprazine, nifurtoinol, nitrofurantoin), quinolones and analogs (e.g., cinoxacin, ciprofloxacin, clinafloxacin, difloxacin, enoxacin, fleroxacin, flumequine, grepafloxacin, lomefloxacin, miloxacin, nadifloxacin, nalidixic acid, norfloxacin, ofloxacin, oxolinic acid, pazufloxacin, pefloxacin, pipemidic acid, piromidic acid, rosoxacin, rufloxacin, sparfloxacin, temafloxacin, tosufloxacin, trovafloxacin), sulfonamides (e.g., acetyl sulfamethoxypyrazine, benzylsulfamide, chloramine-B, chloramine-T, dichloramine T, n²-formylsulfisomidine, n⁴-β-D-glucosylsulfanilamide, mafenide, 4′-(methylsulfamoyl)sulfanilanilide, noprylsulfamide, phthalylsulfacetamide, phthalylsulfathiazole, salazosulfadimidine, succinylsulfathiazole, sulfabenzamide, sulfacetamide, sulfachlorpyridazine, sulfachrysoidine, sulfacytine, sulfadiazine, sulfadicramide, sulfadimethoxine, sulfadoxine, sulfaethidole, sulfaguanidine, sulfaguanol, sulfalene, sulfaloxic acid, sulfamerazine, sulfameter, sulfamethazine, sulfamethizole, sulfamethomidine, sulfamethoxazole, sulfamethoxypyridazine, sulfametrole, sulfamidochrysoidine, sulfamoxole, sulfanilamide, 4-sulfanilamidosalicylic acid, n⁴-sulfanilylsulfanilamide, sulfanilylurea, n-sulfanilyl-3,4-xylamide, sulfanitran, sulfaperine, sulfaphenazole, sulfaproxyline, sulfapyrazine, sulfapyridine, sulfasomizole, sulfasymazine, sulfathiazole, sulfathiourea, sulfatolamide, sulfisomidine, sulfisoxazole) sulfones (e.g., acedapsone, acediasulfone, acetosulfone sodium, dapsone, diathymosulfone, glucosulfone sodium, solasulfone, succisulfone, sulfanilic acid, p-sulfanilylbenzylamine, sulfoxone sodium, thiazolsulfone), and others (e.g., clofoctol, hexedine, methenamine, methenamine anhydromethylene citrate, methenamine hippurate, methenamine mandelate, methenamine sulfosalicylate, nitroxoline, taurolidine, xibomol).

Non-limiting examples of immunosuppressive agents include dexamethasone, cyclosporin A, azathioprine, brequinar, gusperimus, 6-mercaptopurine, mizoribine, rapamycin, tacrolimus (FK-506), folic acid analogs (e.g., denopterin, edatrexate, methotrexate, piritrexim, pteropterin, Tomudex®, trimetrexate), purine analogs (e.g., cladribine, fludarabine, 6-mercaptopurine, thiamiprine, thiaguanine), pyrimidine analogs (e.g., ancitabine, azacitidine, 6-azauridine, carmofur, cytarabine, doxifluridine, emitefur, enocitabine, floxuridine, fluorouracil, gemcitabine, tegafur), fluocinolone, triaminolone, anecortave acetate, fluorometholone, medrysone, and prednisolone.

Non-limiting examples of antifungal agents include polyenes (e.g., amphotericin B, candicidin, dermostatin, filipin, fungichromin, hachimycin, hamycin, lucensomyc in, mepartricin, natamycin, nystatin, pecilocin, perimycin), azaserine, griseofulvin, oligomycins, neomycin undecylenate, pyirolnitrin, siccanin, tubercidin, viridin, allylamines (e.g., butenafine, naftifine, terbinafine), imidazoles (e.g., bifonazole, butoconazole, chlordantoin, chlormidazole, cloconazole, clotrimazole, econazole, enilconazole, fenticonazole, flutrimazole, isoconazole, ketoconazole, lanoconazole, miconazole, omoconazole, oxiconazole nitrate, sertaconazole, sulconazole, tioconazole), thiocarbamates (e.g., tolciclate, tolindate, tolnaftate), triazoles (e.g., fluconazole, itraconazole, saperconazole, terconazole), acrisorcin, amorolfine, biphenamine, bromosalicylchloranilide, buclosamide, calcium propionate, chlorphenesin, ciclopirox, cloxyquin, coparaffinate, diamthazole dihydrochloride, exalamide, flucytosine, halethazole, hexetidine, loflucarban, nifuratel, potassium iodide, propionic acid, pyrithione, salicylanilide, sodium propionate, sulbentine, tenonitrozole, triacetin, ujothion, undecylenic acid, and zinc propionate.

Non-limiting examples of antiviral agents include acyclovir, carbovir, famciclovir, ganciclovir, penciclovir, and zidovudine.

Non-limiting examples of antiprotozoal agents include pentamidine isethionate, quinine, chloroquine, and mefloquine.

In one aspect, the amount of a therapeutic agent is in the range from 0.001 to 10 percent (or alternatively, from 0.005 to 5, or 0.01 to 2, or 0.01 to 1, or 0.01 to 0.5, or 0.1 to 0.5, or 0.1 to 1, or 0.1 to 2, or 0.5 to 2, or 0.5 to 5 percent) by weight of the pharmaceutical composition.

In one embodiment, the pharmaceutical component comprises a fluoroquinolone having Formula I (a new-generation fluoroquinolone antibacterial agent, disclosed in U.S. Pat. No. 5,447,926, which is incorporated herein by reference).

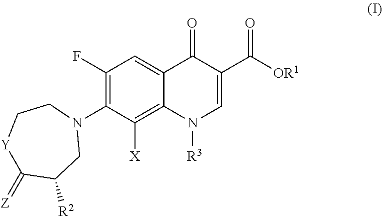

wherein R¹ is selected from the group consisting of hydrogen, unsubstituted C₁-C₅ alkyl groups, substituted C₁-C₅ alkyl groups, C₃-C₇ cycloalkyl groups, unsubstituted C₅-C₂₄ aryl groups, substituted C₅-C₂₄ aryl groups, unsubstituted C₅-C₂₄ heteroaryl groups, and substituted C₅-C₂₄ heteroaryl groups; R² is selected from the group consisting of hydrogen, unsubstituted amino group, and amino groups substituted with one or two C₁-C₅ alkyl groups; R³ is selected from the group consisting of hydrogen, unsubstituted C₁-C₅ alkyl groups, substituted C₁-C₅ alkyl groups, C₃-C₇ cycloalkyl groups, unsubstituted C₁-C₅ alkoxy groups, substituted C₁-C₅ alkoxy groups, unsubstituted C₅-C₂₄ aryl groups, substituted C₅-C₂₄ aryl groups, unsubstituted C₅-C₂₄ heteroaryl groups, substituted C₅-C₂₄ heteroaryl groups, unsubstituted C₅-C₂₄ aryloxy groups, substituted C₅-C₂₄ aryloxy groups, unsubstituted C₅-C₂₄ heteroaryloxy groups, and substituted C₅-C₂₄ heteroaryloxy groups; X is selected from the group consisting of halogen atoms; Y is selected from the group consisting of CH₂, O, S, SO, SO₂, and NR⁴, wherein R⁴ is selected from the group consisting of hydrogen, unsubstituted C₁-C₅ alkyl groups, substituted C₁-C₅ alkyl groups, and C₃-C₇ cycloalkyl groups; and Z is selected from the group consisting of oxygen and two hydrogen atoms; and wherein when a group is substituted, a substituent is selected from the group consisting of hydroxyl, amino, halogen, C₁-C₅ alkyl, C₁-C₅ alkoxy, C₁-C₅ halogenated alkyl, SO₂, and thiol.

In another embodiment, the pharmaceutical component comprises a fluoroquinolone having Formula II.

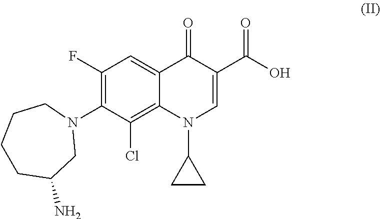

((R)-(+)-7-(3-amino-2,3,4,5,6,7-hexahydro-1H-azepin-1-yl)-8-chloro-1-cyclopropyl-6-fluoro-1,4-dihydro-4-oxoquinoline-3-carboxylic acid).

In still another embodiment, the pharmaceutical component comprises a glucocorticoid receptor agonist having Formulae III or IV, as disclosed in US Patent Application Publication 2006/0116396, which is incorporated herein by reference.

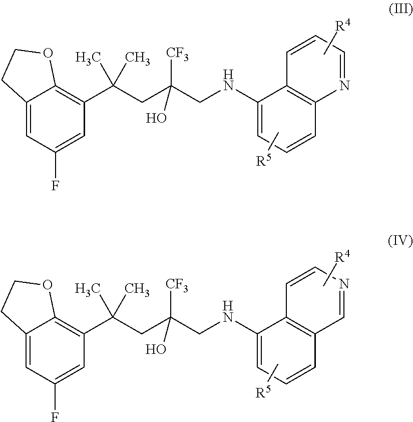

wherein R⁴ and R⁵ are independently selected from the group consisting of hydrogen, halogen, cyano, hydroxy, C₁-C₁₀ (alternatively, C₁-C₅ or C₁-C₃) alkoxy groups, unsubstituted C₁-C₁₀ (alternatively, C₁-C₅ or C₁-C₃) linear or branched alkyl groups, substituted C₁-C₁₀ (alternatively, C₁-C₅ or C₁-C₃) linear or branched alkyl groups, unsubstituted C₃-C₁₀ (alternatively, C₃-C₆ or C₃-C₅) cyclic alkyl groups, and substituted C₃-C₁₀ (alternatively, C₃-C₆ or C₃-C₅) cyclic alkyl groups, wherein when a group is substituted, a substituent is selected from the group consisting of hydroxyl, amino, halogen, C₁-C₅ alkyl, C₁-C₅ alkoxy, C₁-C₅ halogenated alkyl, and thiol.

In yet another embodiment, the pharmaceutical component comprises a glucocorticoid receptor agonist having Formula V (a species of compound having Formula III).

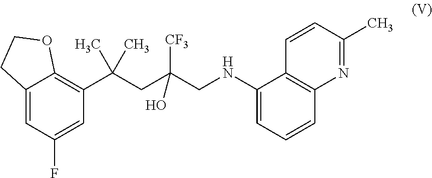

In another embodiment, the therapeutic agent is loteprednol etabonate, an anti-inflammatory agent, having Formula VI.

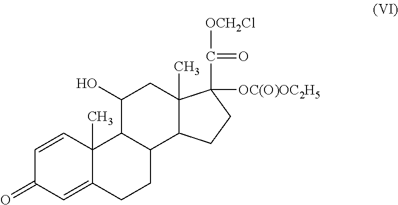

A pharmaceutical composition of the present invention can further comprise a material selected from the group consisting of buffer, tonicity-adjusting agent, viscosity-adjusting agent, pH adjusting agents, antioxidants, chelating agents, and surfactants, and other agents as desired.

An ophthalmic solution of the present invention can be formulated in a physiologically acceptable buffer to regulate pH and tonicity in a range compatible with ophthalmic uses and with any active ingredients present therein. Non-limiting examples of physiologically acceptable buffers include phosphate buffer; a Tris-HCl buffer (comprising tris(hydroxymethyl)aminomethane and HCl); buffers based on HEPES (N-{2-hydroxyethyl}peperazine-N′-{2-ethanesulfonic acid}) having pKₐ of 7.5 at 25° C. and pH in the range of about 6.8-8.2; BES (N,N-bis{2-hydroxyethyl}2-aminoethanesulfonic acid) having pKₐ of 7.1 at 25° C. and pH in the range of about 6.4-7.8; MOPS (3-{N-morpholino}propanesulfonic acid) having pKₐ of 7.2 at 25° C. and pH in the range of about 6.5-7.9; TES (N-tris{hydroxymethyl}-methyl-2-aminoethanesulfonic acid) having pKₐ of 7.4 at 25° C. and pH in the range of about 6.8-8.2; MOBS (4-{N-morpholino}butanesulfonic acid) having pKₐ of 7.6 at 25° C. and pH in the range of about 6.9-8.3; DIPSO (3-(N,N-bis{2-hydroxyethyl}amino)-2-hydroxypropane)) having pKₐ of 7.52 at 25° C. and pH in the range of about 7-8.2; TAPSO (2-hydroxy-3{tris(hydroxymethyl)methylamino}-1-propanesulfonic acid)) having pKₐ of 7.61 at 25° C. and pH in the range of about 7-8.2; TAPS ({(2-hydroxy-1,1-bis(hydroxymethyl)ethyl)amino}-1-propanesulfonic acid)) having pKₐ of 8.4 at 25° C. and pH in the range of about 7.7-9.1; TABS (N-tris(hydroxymethyl)methyl-4-aminobutanesulfonic acid) having pKₐ of 8.9 at 25° C. and pH in the range of about 8.2-9.6; AMPSO (N-(1,1-dimethyl-2-hydroxyethyl)-3-amino-2-hydroxypropanesulfonic acid)) having pKₐ of 9.0 at 25° C. and pH in the range of about 8.3-9.7; CHES (2-cyclohexylamino)ethanesulfonic acid) having pKₐ of 9.5 at 25° C. and pH in the range of about 8.6-10.0; CAPSO (3-(cyclohexylamino)-2-hydroxy-1-propanesulfonic acid) having pKₐ of 9.6 at 25° C. and pH in the range of about 8.9-10.3; or CAPS (3-(cyclohexylamino)-1-propane sulfonic acid) having pKₐ of 10.4 at 25° C. and pH in the range of about 9.7-11.1.

While the buffer itself is a “tonicity adjusting agent” and a “pH adjusting agent” that broadly maintains the ophthalmic solution at a particular ion concentration and pH, additional “tonicity adjusting agents” can be added to adjust the final tonicity of the solution. Non-limiting examples of tonicity-adjusting agents include, but are not limited to, mannitol, sorbitol, urea, propylene glycol, and glycerin. Also, various salts, including halide salts of a monovalent cation (e.g., NaCl or KCl) can be utilized.

The tonicity adjusting agent, when present, can be in a concentration ranging from about 0.01 to about 10, or from about 0.01 to about 7, or from about 0.01 to about 5, or from about 0.1 to about 2, or from about 0.1 to about 1 percent by weight. In some embodiments where a tonicity adjusting agent is present the solution can contain a single agent or a combination of different tonicity adjusting agents. Typically, the tonicity of a formulation of the present invention is in the range from about 200 to 400 mOsm/kg. Alternatively, the tonicity of a formulation of the present invention is in the range from about 220 to 400 mOsm/kg, or from about 220 to 350 mOsm/kg, or from about 220 to 300 mOsm/kg, or from about 250 to 350 mOsm/kg, or from about 250 to 300 mOsm/kg, or from about 240 to 280 mOsm/kg. For certain applications, such as relief of dry eye symptoms or treatment of ocular inflammation, an ophthalmic formulation of the present invention may be desirably hypotonic, such as having tonicity in the range from about 200 to about 270 mOsm/kg, or from about 250 to about 270 mOsm/kg.

Non-limiting examples of viscosity-adjusting agents include synthetic and natural polymers such as poly(acrylic acid) (e.g., the lightly cross-linked poly(acrylic acid) known as Carbopol®, carbomer, or polycarbophil), polysaccharides (e.g., alginic acid, gellan gum, β-glucan, guar gum, gum arabic (a mixture of arabinogalactan ologosaccharides, polysaccharides, and glycoproteins), locust bean gum, pectin, xanthan gum, hyaluronic acid, carboxymethyl starch, carboxymethyl dextran, dextran sulfate, carboxymethyl chitosan, or chondroitin sulfate (e.g., chondroitin sulfate A, chondroitin sulfate B, or chondroitin sulfate C), carrageenan, or curdlan gum), derivatives of cellulose (e.g., carboxymethyl cellulose, methyl cellulose, hydroxypropyl cellulose, hydroxypropyl methyl cellulose, or hydroxyethyl methyl cellulose), or salts thereof. It should be understood that some of the polysaccharides enumerated above may not have naturally occurring salts. Various polyethylene glycols (such as PEG-1000, PEG-3350, PEG-4000, PEG-8000, PEG-10000) may also be considered to be viscosity-adjusting agent.

The amount of a viscosity-adjusting agent may be selected to give the pharmaceutical composition a viscosity in the range from about 2 to about 2,000 centipoises (or mPa·s), as measured by a Brookfield viscometer (Model RVDV III) at 25° C. and a shear rate of 1-7 sec⁻¹, with a CPE-40 spindle. The amount of added viscosity-adjusting agent to achieve a certain viscosity can be easily determined experimentally.

Non-limiting examples of anti-oxidants include ascorbic acid (vitamin C) and its salts and esters; tocopherols (such as α-tocopherol) and tocotrienols (vitamin E), and their salts and esters (such as vitamin E TGPS (D-α-tocopheryl polyethylene glycol 1000 succinate)); glutathione; lipoic acid; uric acid; butylated hydroxyanisole (“BHA”); butylated hydroxytoluene (“BHT”); tertiary butylhydroquinone (“TBHQ”); and polyphenolic anti-oxidants (such as gallic acid, cinnanmic acid, flavonoids, and their salts, esters, and derivatives). In some embodiments, the anti-oxidant comprises ascorbic acid (vitamin C) and its salts and esters; tocopherols (such as α-tocopherol) and tocotrienols (vitamin E), and their salts and esters; BHT; or BHA.

In still another embodiment, the amount of an anti-oxidant in a pharmaceutical formulation of the present invention is in the range from about 0.0001 to about 5 percent by weight of the formulation. Alternatively, the amount of an anti-oxidant is in the range from about 0.001 to about 3 percent, or from about 0.001 to about 1 percent, or from greater than about 0.01 to about 2 percent, or from greater than about 0.01 to about 1 percent, or from greater than about 0.01 to about 0.7 percent, or from greater than about 0.01 to about 0.5 percent, or from greater than about 0.01 to about 0.2 percent, or from greater than about 0.01 to about 0.1 percent, or from greater than about 0.01 to about 0.07 percent, or from greater than about 0.01 to about 0.05 percent, or from greater than about 0.05 to about 0.15 percent, or from greater than about 0.03 to about 0.15 percent by weight of the solution, or from greater than about 0.1 to about 1 percent, or from greater than about 0.1 to about 0.7 percent, or from greater than about 0.1 to about 0.5 percent, or from greater than about 0.1 to about 0.2 percent, or from greater than about 0.1 to about 0.15 percent.

Non-limiting chelating agents include compounds having Formula VII, VIII, or IX.

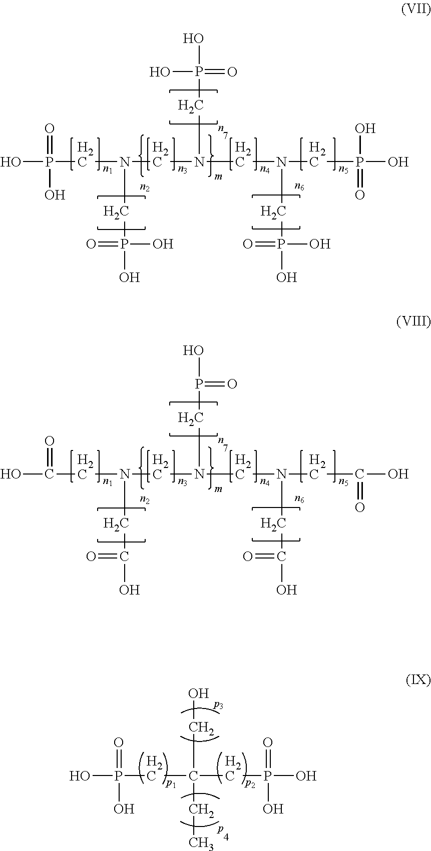

wherein n₁, n₂, n₃, n₄, n₅, n₆, and n₇ are integers independently in the range from 1 to 4, inclusive; m is an integer in the range from 1 to 3, inclusive; p₁, p₂, p₃, and p₄ are independently selected from 0 and integers in the range from 1 to 4, inclusive.

In some embodiments, the chelating agent comprises a compound selected from the group consisting of ethylenediaminetetraacetic acid (“EDTA”), diethylenetriaminepentakis(methylphosphonic acid), etidronic acid, pharmaceutically acceptable salts thereof, and mixtures thereof.

In some other embodiments, the chelating agent comprises tetrasodium salt of etidronic acid (also known as “HAP”, which is available as 30% solution).

In still some other embodiments, the chelating agent comprise EDTA sodium salt.

Ophthalmic solutions of the present invention also can comprise one or more surfactants. Suitable surfactants can include cationic, anionic, non-ionic or amphoteric surfactants. Preferred surfactants are neutral or nonionic surfactants. Non-limiting examples of surfactants suitable for a formulation of the present invention include polyethylene glycol (“PEG,” such as PEG-400, PEG-800, PEG-1000, PEG-3350, PEG-4000, PEG-8000, PEG-10000), polysorbates (such as polysorbate 80 (polyoxyethylene sorbitan monooleate), polysorbate 60 (polyoxyethylene sorbitan monostearate), polysorbate 20 (polyoxyethylene sorbitan monolaurate), commonly known by their trade names of Tween® 80, Tween® 60, Tween® 20), poloxamers (synthetic block polymers of ethylene oxide and propylene oxide, such as those commonly known by their trade names of Pluronic®; e.g., Pluronic® F127 or Pluronic® F108)), poloxamines (synthetic block polymers of ethylene oxide and propylene oxide attached to ethylene diamine, such as those commonly known by their trade names of Tetronic®; e.g., Tetronic® 1508 or Tetronic® 908, etc.), other nonionic surfactants such as Brij®, Myrj®, and long chain fatty alcohols (i.e., oleyl alcohol, stearyl alcohol, myristyl alcohol, docosohexanoyl alcohol, etc.) with carbon chains having about 12 or more carbon atoms (e.g., such as from about 12 to about 24 carbon atoms). Such compounds are delineated in Martindale, 34^{th} ed., pp 1411-1416 (Martindale, “The Complete Drug Reference,” S. C. Sweetman (Ed.), Pharmaceutical Press, London, 2005) and in Remington, “The Science and Practice of Pharmacy,” 21^{st} Ed., pp 291 and the contents of chapter 22, Lippincott Williams & Wilkins, New York, 2006. The concentration of a non-ionic surfactant, when present, in a composition of the present invention can be in the range from about 0.001 to about 5 weight percent (or alternatively, from about 0.01 to about 4, or from about 0.01 to about 2, or from about 0.01 to about 1 weight percent).

In addition to those classes of ingredients disclosed above, a pharmaceutical formulation, such as an ophthalmic solution, of the present invention can further comprise one or more other ingredients, such as vitamins (other than those disclose hereinabove), or other ingredients that provide added health benefits to the users. Where an ophthalmic solution is intended for contact-lens care, it can comprise other known components that are generally used for cleaning and maintenance of contact lenses, as long as these components are compatible with other ingredients in the formulation. In one embodiment, a contact-lens care solution can comprise microabrasives (e.g., polymer microbeads).

In one embodiment, a pharmaceutical composition of the present invention comprises a therapeutic agent, a buffer, a non-ionic surfactant, a pharmaceutically acceptable preservative, and a material selected from the group consisting of D-glucose, sucrose, maltose, D-mannose, trehalose, glutamic acid, mixtures thereof, and combinations thereof, wherein the pharmaceutical composition has an enhanced preservative efficacy against a spore-forming microorganism.

In another embodiment, a pharmaceutical composition of the present invention comprises a therapeutic agent, a buffer, a non-ionic surfactant, a pharmaceutically acceptable preservative, a tonicity-adjusting agent, an anti-oxidant, boric acid, a chelating agent, and a material selected from the group consisting of D-glucose, sucrose, maltose, D-mannose, trehalose, glutamic acid, mixtures thereof, and combinations thereof, wherein the pharmaceutical composition has an enhanced preservative efficacy against a spore-forming microorganism.

In still another embodiment, a pharmaceutical composition of the present invention consists, or consists essentially, of a therapeutic agent, a buffer, a non-ionic surfactant, a pharmaceutically acceptable preservative, a tonicity-adjusting agent, an anti-oxidant, boric acid, a chelating agent, and a material selected from the group consisting of D-glucose, sucrose, maltose, D-mannose, trehalose, glutamic acid, mixtures thereof, and combinations thereof, wherein the pharmaceutical composition has an enhanced preservative efficacy against a spore-forming microorganism.

In some embodiments, the therapeutic agent is selected from the group consisting of compounds having Formulae I-V and mixtures thereof.

In yet another embodiment, a pharmaceutical composition of the present invention consists, or consists essentially, of Compound V, PEG-3350, polysorbate 80, HPMC (hydroxypropylmethylcellulose), a phosphate buffer, D-glucose or sucrose, glycerin, sodium thiosulfate, BHT, polyaminopropyl biguanide (“PAPB,” also known as polyhexamethylene biguanide or PHMB), polyquaternium-1, and EDTA.

Exemplary concentrations of the components of such a composition are shown in Table 2.

TABLE 2

| | | Example 1 Preferred | | |
|---|---|---|---|---|
| | | Concentration | | |
| | Concentration | (wt % except | Comparative | Comparative |
| Ingredient | Range (wt %) | where indicated) | Example 1 | Example 2 |
| PEG-3350 NF | 0.5-20 | 10 | 10 | 10 |
| Polysorbate 80 NF | 0.1-5 | 1 | 1 | 1 |
| HPMC E15LV | 0.05-3 | 1 | 0 | 0.5 |
| Boric acid NF | 0.05-2 | 0.5 | 0.54 | 0.62 |
| D-Glucose or sucrose | 0.1-3 | 0.5 | 0 | 0 |
| Glycerin | 0-3 | 0 | 0 | 0.2 |
| Sodium phosphate dibasic | q.s. for desired | 0.142 | 0.182 | 0.129 |
| anhydrous | buffer pH | | | |
| Sodium phosphate | q.s. for desired | 0 | 01 | 0.012 |
| monobasic monohydrate | buffer pH | | | |
| Sodium thiosulfate | 0.01-0.5 | 0.05 | 0 | 0 |
| pentahydrate | | | | |
| BHT NF | 0-1 | 0 | 0.01 | 0.01 |
| PAPB HCl, 20% solution | 0-30 ppm | 0 | 5 ppm | 0 |
| Polyquaternium-1 | 1-30 ppm | 4 ppm | 0 | 10 ppm |
| EDTA dehydrate | 0-1 | 0 | 0.011 | 0.011 |
| pH | 5.5-8 | 7.4 | 7.1 | 7.4 |
| Osmolality, mOsm/kg | 200-400 | 300 | 290 | 300 |
| Viscosity, cp or MPa · s | 2-2000 | 2-500 | No data | No data |

The preservative efficacy of the preferred composition and the comparative Examples 1 and 2 were tested according to the procedure disclosed herein above. The results are shown in Table 3.

TABLE 3
Preservative Efficacy

| | | Compara- | Compara- | Example 1 Preferred |
|---|---|---|---|---|
| | | tive | tive | Composition of |
| Product | | Example 1 | Example 2 | Present Invention |
| Lot | | C080203A | C081102 | RPG-091001-10 |
| Preservative Efficacy | | Log₁₀ Reduction | | |
| Staphylococcus | Day 7 | >4.7 | >4.8 | >4.8 |
| aureus | Day 14 | >4.7 | >4.8 | >4.8 |
| | Day 28 | >4.7 | >4.8 | >4.8 |
| Pseudomonas | Day 7 | >4.8 | >4.7 | >5.0 |
| aeruginosa | Day 14 | >4.8 | >4.7 | >5.0 |
| | Day 28 | >4.8 | >4.7 | >5.0 |
| Escherichia | Day 7 | >4.8 | >4.8 | >4.8 |
| coli | Day 14 | >4.8 | >4.8 | >4.8 |
| | Day 28 | >4.8 | >4.8 | >4.8 |
| Candida | Day 7 | >4.9 | >4.8 | >4.8 |
| albicans | Day 14 | >4.9 | >4.8 | >4.8 |
| | Day 28 | >4.9 | >4.8 | >4.8 |
| Asperillus | Day 7 | 0.8 | 0.6 | 2.1 |
| brasilensis | Day 14 | 0.8 | 0.5 | 2.9 |
| | Day 28 | 1.0 | 1.2 | 2.9 |

| | | | |
|---|---|---|---|
| Test Disposition | Passes | Passes | Passes USP, |
| | USP | USP | EP-A, and EP-B |

Thus, a composition of the present invention having sucrose has an enhanced preservative efficacy against the spore-forming fungus A. brasiliensis, which enhanced preservative efficacy meets the most stringent criteria of EP-A.

Other compositions were also prepared and tested. To produce these compositions, sucrose in the composition of Example 1 was replaced with L-glucose, D-mannose, maltose, trehalose, or D-fructose, each in the concentration as shown in Table 4. The results of the preservative efficacy testing against A. brasiliensis (a spore-forming fungus) are also shown in Table 4.

TABLE 4
Examples of Replacing Sucrose with other Saccharides

| Example | 1 | 2 | 3 | 4 | 5 |
|---|---|---|---|---|---|
| Formulation | 2593-YH- | 2593-YH- | 2593-YH- | 2593-YH- | 2593-YH- |
| No. | 290-1 | 290-2 | 290-3 | 290-4 | 290-5 |
| Additive | L-glucose | D-mannose | maltose | trehalose | D-fructose |
| Tested | | | | | |

Log₁₀ Reduction

| | | | | | |
|---|---|---|---|---|---|
| Day 7 | 0.9 | 2.0 | 2.3 | 1.7 | 1.1 |
| Day 14 | 0.8 | 2.0 | 1.7 | 1.4 | 1.0 |
| Day 28 | 0.9 | 2.8 | 1.9 | 2.2 | 1.6 |
| Test | Does not | Passes EP-A | Passes EP-A | Passes | Passes |
| Disposition | pass | and EP-B | and EP-B | EP-B | EP-B |

Thus, a composition of the present invention having D-mannose, maltose, trehalose, or D-fructose has an enhanced preservative efficacy against the spore-forming fungus A. brasiliensis, which enhanced preservative efficacy meets the stringet criteria EP-B, or the most stringent criteria of EP-A.

Still other compositions were also prepared and tested. To produce these compositions, sucrose in the composition of Example 1 was replaced with D-glucose, and/or the concentration of polyquaternium-1 (“PQ-1”) was increased to 10 ppm, each in the concentration as shown in Table 5. The results of the preservative efficacy testing against A. brasiliensis (a spore-forming fungus) are also shown in Table 5.

TABLE 5
Examples of Replacing Sucrose with Glucose

| Example | 6 | 7 | 8 |
|---|---|---|---|
| Formulation | 2604-MJC- | 2604-MJC- | 2604-MJC- |
| No. | 214-1 | 214-3 | 232-3 |
| Preservative | 10 ppm PQ-1 | 10 ppm PQ-1 + | 10 ppm PQ-1 + |
| Tested | without | 0.2% | 0.5% |
| | saccharide | D-glucose | sucrose |

Log₁₀ Reduction

| | | | |
|---|---|---|---|
| Day 7 | 0.7 | 2.0 | 2.2 |
| Day 14 | 1.0 | 3.1 | 2.5 |
| Day 28 | 1.0 | 3.9 | No data |
| Test | Passes | Passes EP-A | Passes EP-A |
| Disposition | EP-B | and EP-B | and EP-B |

Thus, while 10 ppm PQ-1 passed the EP-B criteria, adding D-glucose or sucrose enhanced the preservative efficacy to meet the more stringent criteria EP-A.

In another embodiment of the present invention, an amount of 0.5% (by weight) sucrose was added to a commercial eye drop solution that comprises moxifloxacin. The commercial eye drop and the solution with 0.5% sucrose were tested for preservative efficacy against the spore-forming A. brasiliensis. The results are shown in Table 6.

TABLE 6
Effect of Adding Sucrose to Moxifloxacin Solution

| | | |
|---|---|---|
| | | Commercial |
| | Commercial | Solution + 0.5% |
| Formulation | Solution | Sucrose |

Log₁₀ Reduction

| | | |
|---|---|---|
| Day 7 | 2.1 | 3.1 |
| Day 14 | 3.5 | 4.1 |

Thus, addition of 0.5% sucrose enhanced the preservative efficacy against spore-forming A. brasiliensis.

Other embodiments of the present invention were produced wherein the therapeutic agent were loteprednol etabonate and tobramycin and are shown in Table 7. All ingredients are in mg/mL.

TABLE 7

| | Comparative | Example | Example | Example |
|---|---|---|---|---|
| Ingredient | Example 3 | 9 | 10 | 11 |
| Loteprednol | 5 | 5 | 5 | 5 |
| etabonate | | | | |
| Tobramycin | 3 | 3 | 3 | 3 |
| Povidone K90 | 18 | 18 | 18 | 18 |
| Hyrpomellose | 0 | 0 | 10 | 10 |
| E15LV | | | | |
| EDTA disodium | 0.1 | 0.1 | 0.1 | 0.1 |
| dehydrate | | | | |
| Tyloxapol | 3 | 3 | 3 | 3 |
| Boric acid | 0 | 5 | 5 | 5 |
| Sucrose NF | 0 | 5 | 5 | 5 |
| Glycerin | 23.25 | 0 | 0 | 8.8 |
| Propylene | 0 | 10 | 10 | 10 |
| glycol | | | | |
| Benzakonium | 0.2 | 0 | 0 | 0 |
| chloride NF, | | | | |
| 50% solution | | | | |
| Sulfuric acid | q.s. to | 0 | 0 | 0 |
| | pH 5.5 | | | |
| Acetic acid | 0 | q.s. to | q.s. to | q.s. to |
| | | 6.5 | 6.5 | 6.5 |
| Purified water | q.s. to | q.s. to | q.s. to | q.s. to |
| | 1 mL | 1 mL | 1 mL | 1 mL |
| pH | 5.3-5.6 | 6.3-6.6 | 6.3 | 6.6 |
| Osmolality, | 290 | 275 | 280 | 300 |
| mOsm/kg | | | | |
| Viscosity, cp or | 1-3 | 1-3 | 10-15 | 10-15 |
| mPa . s | | | | |

In other embodiments of the present invention, sucrose was added to Besivance®, a commercial ophthalmic suspension prescribed for the treatment of bacterial conjunctivitis. Results of the preservative efficacy testing against spore-forming A. brasiliensis showed that with the added sucrose, these compositions met the most stringent criteria EP-A.

In another aspect, the present invention provides a method for making a pharmaceutical formulation having enhanced preservative efficacy against spore-forming microorganisms. The method comprises adding a pharmaceutically acceptable preservative and a material selected from the group consisting of D-glucose, sucrose, maltose, D-mannose, trehalose, glutamic acid, mixtures thereof, and combinations thereof, to a pharmaceutical formulation.

In still another aspect, the method comprises adding a pharmaceutically acceptable preservative and a material selected from the group consisting of D-glucose, sucrose, maltose, D-mannose, trehalose, glutamic acid, mixtures thereof, and combinations thereof, to a pharmaceutical formulation, wherein the pharmaceutical formulation comprises, consists of, or consists essentially of, a therapeutic agent, a buffer, a tonicity-adjusting agent, a viscosity-adjusting agent, a pH-adjusting agent, a chelating agent, and a surfactant.

In yet another aspect, the method comprises adding a pharmaceutically acceptable preservative and a material selected from the group consisting of D-glucose, sucrose, maltose, D-mannose, trehalose, glutamic acid, mixtures thereof, and combinations thereof, to a pharmaceutical formulation, wherein the pharmaceutical formulation comprises, consists of, or consists essentially of, a buffer, a tonicity-adjusting agent, a viscosity-adjusting agent, a pH-adjusting agent, a chelating agent, an anti-oxidant, and a surfactant. Non-limiting examples of these materials are disclosed herein above.

The method comprises: (1) adding all said materials together, and (2) mixing them to substantial uniformity.

The method can also comprises: (1) adding and mixing some materials together to produce a first mixture; and (2) adding the remaining materials to the first mixture while mixing continues to produce the composition.

The method can also comprises: (1) adding and mixing some materials together to produce a first mixture; (2) adding and mixing the remaining materials together to produce a second mixture; and (3) combining the first mixture the second mixture while mixing continues to produce the composition.

Further non-limiting embodiments of the present invention are shown in the following tables.

### EXAMPLE 12

### Ophthalmic Formulation with Anti-Inflammatory Drug

The following ingredients are combined to produce such a formulation.

| Ingredient | % w/w |
|---|---|
| Sodium Borate | 0.02 |
| Boric Acid | 0.5 |
| Glycerin | 0.6 |
| Propylene Glycol | 0.6 |
| Sodium Alginate (Protanal LF200M) | 0.25 |
| Urea Hydrogen Peroxide | 0.1 |
| HAP (30%) | 0.05 |
| BHA | 0.01 |
| Diclofenac Sodium | 0.5 |
| D-glucose | 0.5 |
| Purified water | q.s. to 100 |

### EXAMPLE 13

### Ophthalmic Formulation for Treating or Controlling High Intraocular Pressure

The following ingredients are combined to produce an exemplary formulation for treating or controlling high intraocular pressure.

| Ingredient | % w/w |
|---|---|
| Sodium Borate | 0.05 |
| Boric Acid | 0.6 |
| Glycerin | 0.75 |
| Propylene Glycol | 0.3 |
| Sodium Alginate (Protanal LF200M) | 0.3 |
| Urea Hydrogen Peroxide | 0.07 |
| HAP (30%) | 0.1 |
| BHA | 0.01 |
| Timolol Maleate | 0.5 |
| Dorzolamide hydrochloride | 2 |
| Sucrose | 0.7 |
| Purified water | q.s. |

### EXAMPLE 14

### Ophthalmic Formulation for Treating or Controlling Eye Infection

The following ingredients are combined to produce such a formulation.

| Ingredient | % w/w |
|---|---|
| Sodium Borate | 0.02 |
| Boric Acid | 0.5 |
| Glycerin | 0.6 |
| Propylene Glycol | 0.6 |
| Polycarbophil (a cross-linked | 0.25 |
| poly(acrylic acid)) | |
| PHMB | 0.01 |
| EDTA | 0.05 |
| Moxifloxacin | 0.5 |
| Maltose | 1 |
| Purified water | q.s. |

### EXAMPLE 15

### Ophthalmic Formulation for Treating or Controlling Eye Infection

The following ingredients are combined to produce such a formulation.

| Ingredient | % w/w |
|---|---|
| Sodium Borate | 0.02 |
| Boric Acid | 0.5 |
| Glycerin | 1 |
| Propylene Glycol | 0.2 |
| Carboxymethyl cellulose | 0.25 |
| PQ-1 | 0.002 |
| HAP (30%) | 0.05 |
| vitamin E TGPS | 0.05 |
| Gatifloxacin | 0.3 |
| Ciprofloxacin | 0.15 |
| Phosphate buffer | q.s. to |
| | pH 6.3-6.8 |
| D-glucose | 0.5 |
| Purified water | q.s. |

### EXAMPLE 16

### Ophthalmic Formulation for Treating or Controlling Eye Infection

The following ingredients are combined to produce such a formulation.

| Ingredient | % w/w |
|---|---|
| Sodium Borate | 0.03 |
| Boric Acid | 0.35 |
| Propylene Glycol | 0.6 |
| HPMC | 0.4 |
| EDTA | 0.05 |
| Benzalkonium chloride | 0.01 |
| Ascorbic acid | 0.05 |
| 7-[(3R)-3-aminohexahydro-1H-azepin- | 0.3 |
| 1-yl]-8-chloro-1-cyclopropyl-6-fluoro- | |
| 1,4-dihydro-4-oxo-3- | |
| quinolinecarboxylic acid | |
| monohydrochloride | |
| Sucrose | 0.5 |
| Phosphate buffer | q.s. to |
| | pH 6.0-7.0 |
| Purified water | q.s. |

### EXAMPLE 17

### Ophthalmic Formulation for Treating or Controlling Eye Allergy

The following ingredients are combined to produce an exemplary formulation for treating or controlling eye allergy.

| Ingredient | % w/w |
|---|---|
| Sodium Borate | 0.06 |
| Boric Acid | 0.7 |
| Propylene Glycol | 0.6 |
| Hyaluronic acid | 0.25 |
| Polyquaternium-42 | 0.005 |
| Diethylenetriaminepentakis(methyl- | 0.05 |
| phosphonic acid) sodium salt | |
| Gallic acid | 0.05 |
| Ketotifen Fumarate | 0.025 |
| D-glucose | 0.5 |
| Phosphate buffer | q.s. to |
| | 7.0-7.6 |
| Purified water | q.s. |

### EXAMPLE 18

### Ophthalmic Formulation for Treating or Controlling Eye Allergy

The following ingredients are combined to produce an exemplary formulation for treating or controlling eye allergy.

| Ingredient | % w/w |
|---|---|
| Sodium Borate | 0,06 |
| Boric Acid | 0.7 |
| Glycerin | 0.6 |
| Propylene Glycol | 0.6 |
| HPMC | 0.4 |
| Sodium perborate 4H2O | 0.07 |
| Cinnamic acid | 0.05 |
| EDTA disodium | 0.05 |
| HAP (30%) | 0.02 |
| Olopatadine Hydrochloride | 0.1 |
| Maltose | 1 |
| Phosphate buffer | q.s. to |
| | pH 7-7.5 |
| Purified water | q.s. |

### EXAMPLE 19

### Ophthalmic Formulation for Treating or Controlling Eye Infection

The following ingredients are combined to produce an exemplary formulation for treating or controlling eye infection.

| Ingredient | % w/w |
|---|---|
| Sodium Borate | 0.06 |
| Boric Acid | 0.7 |
| Glycerin | 0.6 |
| Propylene Glycol | 0.6 |
| CMC | 0.25 |
| Sodium perborate monohydrate | 0.1 |
| Sodium perborate tetrahydrate | 0.05 |
| Vitamin E TPGS | 0.1 |
| HAP (30%) | 0.1 |
| Acyclovir | 0.05 |
| D-glucose | 0.5 |
| Phosphate buffer | q.s. to |
| | pH 6.3-7.4 |
| Purified water | q.s. |

### EXAMPLE 20

### Ophthalmic Formulation for Treating or Controlling Eye Infection

The following ingredients are combined to produce an exemplary formulation for treating or controlling eye infection. The polysaccharide included in this formulation is carboxymethyl cellulose.

| Ingredient | % w/w |
|---|---|
| Sodium Borate | 0.06 |
| Boric Acid | 0.7 |
| Glycerin | 0.8 |
| Propylene Glycol | 0.4 |
| Carboxymethyl cellulose | 0.25 |
| PQ-1 | 0.001 |
| Ascorbic acid | 0.1 |
| HAP (30%) | 0.1 |
| BHA | 0.02 |
| 7-[(3R)-3-aminohexahydro-1H-azepin- | 0.3 |
| 1-yl]-8-chloro-1-cyclopropyl-6-fluoro- | |
| 1,4-dihydro-4-oxo-3- | |
| quinolinecarboxylic acid | |
| monohydrochloride | |
| Phosphate buffer | q.s. to |
| | pH 6.5-7.5 |
| D-glucose | 0.6 |
| Purified water | q.s. |

### EXAMPLE 21

### Ophthalmic Formulation for Treating or Controlling Eye Inflammation

The following ingredients are combined to produce an exemplary formulation for treating or controlling eye inflammation.

| Ingredient | % w/w |
|---|---|
| Sodium Borate | 0.06 |
| Boric Acid | 0.7 |
| Glycerin | 0.6 |
| Propylene Glycol | 0.6 |
| Sodium hyaluronate | 0.25 |
| PQ-1 | 0.1 |
| Resveratrol | 0.075 |
| Ascorbic acid | 0.025 |
| HAP (30%) | 0.1 |
| Loteprednol Etabonate | 0.5 |
| D-glucose | 0.5 |
| Sucrose | 0.2 |
| Phosphate buffer | q.s. to |
| | pH 6.5-7.5 |
| Purified water | q.s. |

### EXAMPLE 22

### Ophthalmic Formulation for Treating or Controlling Eye Inflammation

The following ingredients are combined to produce an exemplary formulation for treating or controlling eye inflammation.

| Ingredient | % w/w |
|---|---|
| Sodium Borate | 0.06 |
| Boric Acid | 0.7 |
| Glycerin | 0.3 |
| Propylene Glycol | 0.8 |
| Chondroitin sulfate | 0.25 |
| Sodium perborate tetrahydrate | 0.1 |
| Diethylenetriaminepentakis(methyl- | 0.05 |
| phosphonic acid) sodium salt | |
| BHA | 0.05 |
| Loteprednol Etabonate | 0.5 |
| Tobramycin | 0.3 |
| D-glucose | 0.6 |
| Phosphate buffer | q.s. to |
| | 6.5-7.5 |
| Purified water | q.s. |

### EXAMPLE 23

### Ophthalmic Formulation for Treating or Controlling Eye Inflammation

The following ingredients are combined to produce an exemplary formulation for treating or controlling eye inflammation.

| Ingredient | % w/w |
|---|---|
| Sodium Borate | 0.06 |
| Boric Acid | 0.7 |
| Mannitol | 0.6 |
| Propylene Glycol | 0.6 |
| Carboxymethyl dextran | 0.25 |
| Urea Hydrogen Peroxide | 0.1 |
| Vitamin E TPGS | 0.05 |
| HAP (30%) | 0.05 |
| Dexamethasone | 0.1 |
| D-glucose | 0.6 |
| Phosphate buffer | q.s. to |
| | pH 6.5-7.5 |
| Purified water | q.s. |

### EXAMPLE 24

### Ophthalmic Formulation for Treating or Controlling Intraocular Pressure

The following ingredients are combined to produce an exemplary formulation for treating or controlling intraocular pressure.

| Ingredient | % w/w |
|---|---|
| Sodium Borate | 0.06 |
| Boric Acid | 0.7 |
| Sorbitol | 0.6 |
| Propylene Glycol | 0.6 |
| Sodium Alginate | 0.25 |
| PHMB | 0.005 |
| Vitamin E TPGS | 0.1 |
| BHA | 0.075 |
| Brimonidine tartrate | 2 |
| Timolol maleate | 0.5 |
| D-glucose | 0.7 |
| D-mannose | 0.2 |
| Phosphate buffer | q.s. to |
| | pH 6.5-7.5 |
| Purified water | q.s. |

### EXAMPLE 25

### Formulation Comprising a Second Preservative

The following ingredients are combined to produce an exemplary formulation. This formulation may be used as a vehicle for an ophthalmic active agent or as a contact-lens treating, cleaning, wetting, or storing solution.

| Ingredient | % w/w |
|---|---|
| Sodium Borate | 0.06 |
| Boric Acid | 0.7 |
| Propylene Glycol | 1 |
| Sodium Alginate | 0.4 |
| PQ-1 | 0.05 |
| EDTA disodium | 0.02 |
| HAP (30%) | 0.05 |
| Anthocyanin (anti-oxidant) | 0.06 |
| Stabilized Oxychloro Complex | 0.01 |
| D-glucose | 0.6 |
| Phosphate buffer | q.s. to |
| | pH 6.5-7.5 |
| Purified water | q.s. |

In another aspect, an ophthalmic solution of the present invention comprising a therapeutic agent that can be used to treat ocular conditions such as dry eye, inflammation, allergy, or infection of the eye.

In still another aspect, the present invention provides methods of making and using a pharmaceutical formulation of the present invention. Any of the materials, compounds, and ingredients disclosed herein is applicable for use with or inclusion in any method of the present invention.

In still another aspect, the present invention provides a method for making a pharmaceutical formulation. The method comprises providing a pharmaceutically acceptable preservative and a material selected from the group consisting of D-glucose, sucrose, maltose, D-mannose, trehalose, glutamic acid, mixtures thereof, and combinations thereof, to a pharmaceutical formulation.

In one embodiment, the method comprises: (a) providing an initial formulation; and (b) adding a pharmaceutically acceptable preservative and a material selected from the group consisting of D-glucose, sucrose, maltose, D-mannose, trehalose, glutamic acid, mixtures thereof, and combinations thereof, to the initial formulation to produce the final pharmaceutical formulation. In another embodiment, the method further comprises adding another ingredient selected from the group consisting of therapeutic agents, buffers, tonicity adjusting agents, surfactants, viscosity adjusting agents, and other agents to the pharmaceutical formulation. The therapeutic agents can be selected from the group of anti-inflammatory agents, agents for lowering intraocular pressure, ocular neuroprotectants, antibiotics, immunosuppressive agents, anti-allergic agents, antiviral agents, antifungal agents, antiprotozoal agents, and mixtures thereof. In still another embodiment, the source of hydrogen peroxide comprises a compound that is soluble in an aqueous medium. Non-limiting examples of each of these classes of agents, compounds, and ingredients are disclosed throughout the present specification.

In still another aspect, the method further comprises adding boric acid and a phosphate buffer to the initial formulation.

In still another aspect, the present invention provides a method for providing safety, or comfort, or both to users of a pharmaceutical formulation. The method comprises adding a pharmaceutically acceptable preservative and a material selected from the group consisting of D-glucose, sucrose, maltose, D-mannose, trehalose, glutamic acid, mixtures thereof, and combinations thereof, to a pharmaceutical formulation, wherein the pharmaceutically acceptable preservative is added in an amount that does not result in irritation or discomfort to an average user. In one embodiment, the pharmaceutical formulation is an ophthalmic composition and the irritation or discomfort is detected in an ocular tissue.

In still another aspect, the present invention provides a method for enhancing the preservative efficacy of a pharmaceutical formulation against spore-forming microorganisms. The method comprises adding a pharmaceutically acceptable preservative and a material selected from the group consisting of D-glucose, sucrose, maltose, D-mannose, trehalose, glutamic acid, mixtures thereof, and combinations thereof, to a pharmaceutical formulation.

In one embodiment, the method comprises: (a) providing an initial formulation; and (b) adding a pharmaceutically acceptable preservative and a material selected from the group consisting of D-glucose, sucrose, maltose, D-mannose, trehalose, glutamic acid, mixtures thereof, and combinations thereof, to the initial formulation to produce the final pharmaceutical formulation. In another embodiment, the method further comprises adding another ingredient selected from the group consisting of therapeutic agents, buffers, tonicity adjusting agents, surfactants, viscosity adjusting agents, and other agents to the pharmaceutical formulation. The therapeutic agents can be selected from the group of anti-inflammatory agents, agents for lowering intraocular pressure, ocular neuroprotectants, antibiotics, immunosuppressive agents, anti-allergic agents, antiviral agents, antifungal agents, antiprotozoal agents, and mixtures thereof. In still another embodiment, the source of hydrogen peroxide comprises a compound that is soluble in an aqueous medium. Non-limiting examples of each of these classes of agents, compounds, and ingredients are disclosed throughout the present specification.

In still another aspect, the present invention provides a method for enhancing the preservative efficacy of a pharmaceutical formulation against spore-forming microorganisms. The method comprises adding a pharmaceutically acceptable preservative, boric acid, phosphate, and a material selected from the group consisting of D-glucose, sucrose, maltose, D-mannose, trehalose, glutamic acid, mixtures thereof, and combinations thereof, to a pharmaceutical formulation.

In yet another aspect, the present invention provides a method for enhancing the preservative efficacy of a pharmaceutical formulation against spore-forming microorganisms. The method comprises adding a pharmaceutically acceptable preservative, boric acid or borate buffer, and a material selected from the group consisting of D-glucose, sucrose, maltose, D-mannose, trehalose, glutamic acid, mixtures thereof, and combinations thereof, to a pharmaceutical formulation.

In yet another aspect, the method enhances the preservative efficacy of the formulation against spore-forming microorganisms to meet the EP-A criteria according to the testing method disclosed herein above.

In one embodiment, such microorganisms comprise spore-forming fungi. In another embodiment such microorganisms comprise spore-forming bacteria. In still another embodiment, such microorganisms comprise spore-forming bacteria and fungi.

While specific embodiments of the present invention have been described in the foregoing, it will be appreciated by those skilled in the art that many equivalents, modifications, substitutions, and variations may be made thereto without departing from the spirit and scope of the invention as defined in the appended claims.

What is claimed is:

1. A pharmaceutical formulation comprising a pharmaceutically acceptable preservative and a preservative efficacy-enhancing material selected from the group consisting of D-glucose, sucrose, maltose, D-mannose, trehalose, glutamic acid, mixtures thereof, and combinations thereof, wherein the formulation has enhanced preservative efficacy against a spore-forming microorganism, and wherein the pharmaceutically acceptable preservative is present in an amount that does not result in irritation or discomfort to an average user.

2. The pharmaceutical formulation of claim 1, wherein the pharmaceutically acceptable preservative is present in an amount that alone does not provide a EP-A preservative efficacy criteria.

3. The pharmaceutical formulation of claim 1, wherein said pharmaceutical formulation further comprises boric acid or borate buffer.

4. The pharmaceutical formulation of claim 2, wherein said pharmaceutical formulation further comprises boric acid or borate buffer.

5. The pharmaceutical formulation of claim 3, wherein said pharmaceutical formulation further comprises a phosphate.

6. The pharmaceutical formulation of claim 4, wherein said pharmaceutical formulation further comprises a phosphate.

7. The pharmaceutical formulation of claim 1, further comprising a therapeutic agent.

8. The pharmaceutical formulation of claim 6, further comprising a therapeutic agent.

9. The pharmaceutical formulation of claim 1, wherein the formulation comprises a solution, a suspension, an emulsion, a dispersion, an ointment, or a cream.

10. The pharmaceutical formulation of claim 8, wherein the formulation comprises a solution or a suspension.

11. The pharmaceutical formulation of claim 8, wherein the formulation comprises an aqueous solution.

12. A method for enhancing the preservative efficacy of a pharmaceutical formulation against a spore-forming microorganism, comprising adding a pharmaceutically acceptable preservative, and a preservative efficacy-enhancing material selected from the group consisting of D-glucose, sucrose, maltose, D-mannose, trehalose, glutamic acid, mixtures thereof, and combinations thereof, to the pharmaceutical formulation.

13. The method of claim 12, further comprising adding boric acid or borate buffer to the pharmaceutical formulation.

14. The method of claim 13, further comprising adding phosphate to the pharmaceutical formulation.

15. The method of claim 12, wherein the preservative efficacy-enhancing material is selected from the group consisting of D-glucose, sucrose, and combinations thereof.

16. The method of claim 12, wherein the preservative efficacy-enhancing material comprises D-glucose.

17. The method of claim 12, wherein the preservative efficacy-enhancing material comprises sucrose.

18. The method of claim 12, wherein the formulation meets the EP-A preservative efficacy criteria.

19. The method of claim 12, wherein the formulation meets the EP-A preservative efficacy criteria.

20. The method of claim 12, wherein the formulation meets the EP-A preservative efficacy criteria, wherein the spore-forming microorganism is A. brasiliensis.
